(19) United States
(12) Patent Application Publication
(10) Pub. No.: US 2017/0268059 A1
(43) Pub. Date: Sep. 21, 2017

(54) DNA HYPERMETHYLATION OF PROMOTERS OF TARGET GENES AND CLINICAL DIAGNOSIS AND TREATMENT OF HPV RELATED DISEASE

(71) Applicant: THE JOHNS HOPKINS UNIVERSITY, Baltimore, MD (US)

(72) Inventors: Rafael Enrique Guerrero-Preston, Baltimore, MD (US); David Sidransky, Baltimore, MD (US)

(21) Appl. No.: 15/601,058

(22) Filed: May 22, 2017

Related U.S. Application Data
(63) Continuation of application No. 14/381,604, filed on Aug. 28, 2014.
(60) Provisional application No. 61/603,652, filed on Feb. 27, 2012.

(51) Int. Cl. C12Q 1/68

(52) U.S. Cl. CPC C12Q 1/6883; C12Q 1/6886; C12Q 1/6827; C12Q 2600/154

(57) ABSTRACT

The present invention provides arrays for gene loci that allow diagnosis of cervical cancer in patients who may be asymptomatic or have inconclusive Pap smears or cytology, and allowing earlier diagnosis and treatment of the subject. The present invention also provides methods of determination of a global promoter DNA methylation in a cervical tissue sample from a subject, using a variety of methods which can detect DNA methylation. Further, the invention provides methods of diagnosis of cervical cancer in a subject, by comparing the global promoter DNA methylation in a cervical tissue sample obtained from a subject to the global promoter DNA methylation of standard controls. In addition, the present invention also provides a method of diagnosis of cervical cancer in a subject suspected of having cervical cancer after obtaining a biological sample of cervical tissue comprising DNA from the subject and detecting the amount of promoter methylation on at least one or more DNA target sites selected from the group consisting of ZNF516, INTS1, and FKBP6; and comparing the amount of promoter methylation on at least one or more DNA target sites in the sample of the subject. These methods allow diagnosis of cervical cancer in patients who may be asymptomatic or have inconclusive Pap smears or cytology, and allowing earlier diagnosis and treatment of the subject.

### REFERENCE TO RELATED APPLICATIONS

This application is a Continuation of U.S. patent application Ser. No. 14/381,604, filed Aug. 28, 2014, which is a 35 U.S.C. §371 U.S. national entry of International Application PCT/US2013/027897, having an international filing date of Feb. 27, 2013, which claims the benefit of U.S. Provisional Patent Application No. 61/603,652, filed on Feb. 27, 2012, the content of each of the aforementioned applications is herein incorporated by reference in their entirety.

### STATEMENT OF GOVERNMENTAL INTEREST

This invention was made with government support under grant nos. CA164092, and CA84986, awarded by the National Institutes of Health. The government has certain rights in the invention.

### INCORPORATION-BY-REFERENCE OF MATERIAL SUBMITTED ELECTRONICALLY

The instant application contains a Sequence Listing which has been submitted electronically in ASCII format and is hereby incorporated by reference in its entirety. Said ASCII copy, created on Aug. 15, 2014, is named P11911-03_SL.txt and is 11,645 bytes in size.

### BACKGROUND OF THE INVENTION

Epigenomics refers to the inheritance of information based on gene expression levels that do not entail changes in DNA sequence, as opposed to genetics which refers to information transmitted on the basis of gene sequence. The best understood epigenomic marks include DNA methylation, histone modifications, and micro-RNA (miRNA). Epigenomics has been called the science of change. It is a biological endpoint for endogenous and exogenous factors that determine health and disease.

DNA methylation is one of the most common alterations in human neoplasia, including breast cancer. DNA methylation refers to the addition of a methyl group to the cytosine ring of those cytosines that precede guanosine (CpG dinucleosides) to form methyl cytosine. Detection of changes in DNA methylation may offer an alternative to screening and may offer data for long-term management of women treated for breast cancer.

Cervical cancer is a cellular alteration that originates in the epithelium of the cervix and is initially apparent through slow and progressively evolving precursor lesions (cervical intraepithelial neoplasia (CIN)), which can be grouped into low and high grade squamous intraepithelial lesions (LSIL and HSIL respectively). 50% of HSIL will eventually progress to cervical cancer. Alterations in cell cycle control mediated by human papilloma virus (HPV) oncoproteins are the main molecular mechanism of action in cervical cancer. HPV infection is very common; the life-time risk for productive women is around 80%. However, most women clear the infection, regardless of HPV type, without experiencing adverse health effects. The most frequently involved HPV types in cervical lesions are HPV 16 and 18, which together cause 70% of cervical cancer cases. Oncogenic HPV infection is a necessary, albeit not sufficient, factor for the oncogenic transformation of cervical-epithelial cells. Additional cofactors, such as an effective immune response leading to viral clearance, determine whether HPV infection will lead to cervical cancer.

Cytology screening with the Papanicolau (Pap) test has substantially reduced cervical cancer incidence and mortality where it has been successfully implemented. The Pap test is limited by relatively low sensitivity (55%) for detection of high-grade cervical lesions. More recently, detection of high-risk HPV types has been suggested as a new screening test; however it is associated with lower specificity than the Pap test.

There is currently no methylation biomarker that can be readily translated for cervical cancer screening. An aim of the present invention was to discover novel methylation biomarkers for cervical cancer screening by methylation microarray analysis and to test whether these markers could discriminate between normal and cancerous cervical tissues, both in vitro and in clinical samples.

Therefore, there still exists a need for additional biomarkers to improve cervical cancer screening.

### SUMMARY OF THE INVENTION

In accordance with an embodiment, the present invention provides an array of oligonucleotide probes for identifying methylated promoters of target DNA genes in a sample, comprising one or more oligonucleotide probes that each selectively bind methylated loci in a target DNA gene and a platform; wherein the probes are immobilized on the platform; and wherein at least one or more probes selectively bind methylated promoter target DNA genes selected from the group consisting of GGTLA4, CGB5, FKBP6, TRIM74, ZNF516, MICAL-L2, ZAP701, RGS12, SAP130 and INTS1.

In accordance with an embodiment, the present invention provides a biochip comprising a solid substrate further comprising at least two oligonucleotide probes of any of the arrays described above, which are capable of hybridizing to a target sequence under stringent hybridization conditions and attached at spatially defined address on the substrate.

In accordance with another embodiment, the present invention provides a method for determining the methylation status of one or more target genes in a cervical tissue sample from a subject comprising: a) obtaining a biological sample of comprising DNA from the cervical tissue of the subject; (b) extracting DNA from the sample of a); (c) contacting the DNA from (b) with the any of the arrays described above or the biochip described above; (d) performing an analysis using the array or biochip of c) to determine the methylation of at least one or more target DNA genes obtained from the sample; and (e) comparing the methylation of at least one or more target DNA genes obtained from the sample tissue with the methylation of at least one target DNA gene obtained from a control sample, wherein a detectable increase in the promoter methylation of at least one or more target DNA genes obtained from the sample compared to control wherein when the amount of promoter methylation on at least one or more DNA target genes is greater than the amount of promoter methylation in the control sample, the promoter of the target DNA gene is considered to be methylated.

In accordance with an embodiment, the present invention provides a method of diagnosis of cervical cancer in a subject suspected of having cervical cancer comprising a) obtaining a biological sample of cervical tissue comprising DNA from the subject, b) detecting the amount of promoter methylation on at least one or more DNA target sites selected from the group consisting of ZNF516, INTS1, and FKBP6, and c) comparing the amount of promoter methylation on at least one or more DNA target sites in the sample of the subject to the amount of promoter methylation in a control sample, wherein when the amount of promoter methylation on at least one or more DNA target sites is greater than the amount of promoter methylation in the control sample, the subject is diagnosed as having cervical cancer.

In another embodiment, the present invention provides a method of screening of a subject suspected of having an increased risk of having a cervical neoplasia comprising a) obtaining a biological sample of cervical tissue comprising DNA from the subject, b) detecting the amount of promoter methylation on at least one or more DNA target sites selected from the group consisting of ZNF516, INTS1, and FKBP6, and c) comparing the amount of promoter methylation on at least one or more DNA target sites in the sample of the subject to the amount of promoter methylation in a control sample, wherein when the amount of promoter methylation on at least one or more DNA target sites is greater than the amount of promoter methylation in the control sample, the subject is diagnosed as an increased risk of having a cervical neoplasia.

In a further embodiment, the present invention provides a method of diagnosis of cervical cancer in a subject suspected of having cervical cancer comprising a) obtaining a biological sample of cervical tissue comprising DNA from the subject, b) detecting the amount of global promoter methylation of the DNA from the subject, and c) comparing the amount of global promoter methylation in the sample of the subject to the amount of global promoter methylation in a control sample, wherein when the amount of global promoter methylation of the DNA of the subject is less than the amount of global promoter methylation in the DNA of the control sample, the subject is diagnosed as having cervical cancer.

### BRIEF DESCRIPTION OF THE DRAWINGS

FIG. 1 is a flowchart of the data analysis and integration tasks performed to identify ZNF516, INTS1 and FKBP6 as hypermethylated and down regulated biomarkers in cervical cancer.

FIG. 2A provides scatterplots of qMSP analysis of candidate gene promoters in the Discovery cohort (normal n=19, cancer n=30). The relative level of methylated DNA for each gene in each sample was determined as a ratio of MSP for the amplified gene to β-actin. Red line denotes cut-off value; FIG. 2B provides scatterplots of qMSP analysis of FKBP6, INTS1, and ZNF516 in the Prevalence cohort (normal n=18, cancer n=90). The relative level of methylated DNA for each gene in each sample was determined as a ratio of MSP for the amplified gene to β-actin. The red line denotes the cut-off value.

FIG. 3A shows scatterplots of qMSP analysis of ZNF516 in HPV positive and non-detected normal (n=37) and cervical cancer samples (n=120) from both Discovery and Prevalence cohorts. The relative level of methylated DNA for each gene in each sample was determined as a ratio of MSP for the amplified gene to B-actin. The blue line denotes the cut-off value. Red circles denote cervical cancer samples. Black circles denotes normal cervical mucosa samples; FIG. 3B shows results of separate unadjusted and adjusted logistic regression models fitted to examine the association between clinical diagnosis of cancer and promoter methylation of FKBP6, INTS1 and ZNF516 after controlling for the potential confounding of age and HPV status.

FIG. 4 shows bisulfite sequencing candidate genes in the same samples used to hybridize microarrays. The figure represents CpG methylation density in the promoter regions. Bisulfite sequence analysis results are summarized as filled circles representing methylated CpGs and open circles representing unmethylated CpGs. (The figure shows only the first seven cytosines of the fragment, in six representative samples of the population).

FIG. 5 depicts Methylation Specific PCR (MSP) results in the samples that were hybridized to the microarrays. M: Methylated, U: Unmethylated; Positive Control (C+) 100% Methylated Bisulfite treated DNA (ZymoResearch); PCR product without DNA (blank). (I) Normal Samples; (II) Tumor.

FIG. 6A-6E shows methylation frequency bar charts by histology type: 25 Normal samples, 66 LSIL (Low Squamous Intraepithelial Lesions), 91 HSIL (High Squamous Intraepithelial Lesions) and 39 CC (Tumor). A: GGTLA4, B: FKBP6, C: ZNF516, D: INTS1 and E: Sap130.

FIG. 7A-7F depicts MSP results for A: B-actin (268 bp), B: GGTLA4 (M183, U185 bp), C: FKBP6 (M137, U135 bp), D: ZNF516 (M 241, U 242 bp), E: INTS1 (M 143, U 147 bp) and F: SAP130 (M 189, U 192 bp) by histology type. M: Methylated, U: Unmethylated.

### DETAILED DESCRIPTION OF THE INVENTION

The use of hypermethylated genes as cervical cancer screening and triage biomarkers is advantageous because tissue specific changes in DNA methylation are characteristic of neoplastic cells, regardless of whether they are epigenetic drivers or passengers of the oncogenic process.

The clinical implications of the findings of the present invention are multiple. In southern Chile approximately 40% of the colposcopies and cone-biopsies performed in high-risk cervical cancer clinics turn out to be negative. ZNF516 and FKBP6, and other genes may thus be used to reduce the number of these unnecessary cervical biopsy examinations, without reducing the number of women with premalignant and invasive cervical cancer that receive biopsy examinations.

In accordance with an embodiment, the present invention provides a method of diagnosis of cervical cancer in a subject suspected of having cervical cancer comprising a) obtaining a biological sample of cervical tissue comprising DNA from the subject, b) detecting the amount of promoter methylation on at least one or more DNA target sites selected from the group consisting of ZNF516, INTS1, and FKBP6, and c) comparing the amount of promoter methylation on at least one or more DNA target sites in the sample of the subject to the amount of promoter methylation in a control sample, wherein when the amount of promoter methylation on at least one or more DNA target sites is greater than the amount of promoter methylation in the control sample, the patient is diagnosed as having cervical cancer.

In accordance with another embodiment of the present invention, it will be understood that the term “biological sample” or “biological fluid” includes, but is not limited to, any quantity of a substance from a living or formerly living patient or mammal. Such substances include, but are not limited to, blood, serum, plasma, urine, cells, organs, tissues, bone, bone marrow, lymph, lymph nodes, synovial tissue, chondrocytes, synovial macrophages, endothelial cells, and skin.

It will be understood by those of ordinary skill, that there are a number of ways to detect DNA methylation, and these are known in the art. Examples of preferred methods of detection of methylation of DNA in a sample using the methods of the present invention include the use of qMSP, oligonucleotide methylation tiling arrays, paramagnetic beads linked to MBD2, i.e., BeadChip assays and HPLC/MS methods. Other methods include methylation-specific multiplex ligation-dependent probe amplification (MS-MPLA), bisulfate sequencing, and assays using antibodies to DNA methylation, i.e., ELISA assays.

As used herein, the term “subject suspected of having cervical cancer” or “subject suspected of having an increased risk of having a cervical neoplasia” includes a patient presenting cervical intraepithelial neoplasia (CIN), and/or low grade squamous intraepithelial lesion (LSIL) and/or high grade squamous intraepithelial lesion (HSIL), or any other abnormal Pap smear or cytological test.

As used herein, the term “methylation state” means the detection of one or more methyl groups on a cytidine in a target site of the DNA in the sample.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

“Identical” or “identity” as used herein in the context of two or more nucleic acids or polypeptide sequences may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind. In accordance with one or more embodiments, the term “probe” also means an oligonucleotide which is capable of specifically binding to a CpG locus which can be methylated. The DNA gene target or probes of the present invention are used to determine the methylation status of at least one CpG dinucleotide sequence of at least one target gene as described herein.

“Substantially complementary” used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

A probe is also provided comprising a nucleic acid described herein. Probes may be used for screening and diagnostic methods, as outlined below. The probes may be attached or immobilized to a solid substrate or apparatus, such as a biochip.

The probe may have a length of from 8 to 500, 10 to 100 or 20 to 60 nucleotides. The probe may also have a length of at least 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 nucleotides. The probe may further comprise a linker sequence of from 10-60 nucleotides.

In accordance with one or more embodiments, the arrays of the present invention further comprise at least one randomly-generated oligonucleotide probe sequence used as a negative control; at least one oligonucleotide sequence derived from a housekeeping gene, used as a negative control for total DNA degradation; at least one randomly-generated sequence used as a positive control; and a series of dilutions of at least one positive control sequence used as saturation controls; wherein at least one positive control sequence is positioned on the array to indicate orientation of the array.

A biochip is also provided. The biochip is an apparatus which, in certain embodiments, comprises a solid substrate comprising an attached probe or plurality of probes described herein. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. In an embodiment, two or more probes per target sequence are used. The probes may be capable of hybridizing to target sequences associated with a single disorder.

The probes may be attached to the biochip in a wide variety of ways, as will be appreciated by those in the art. The probes may either be synthesized first, with subsequent attachment to the biochip, or may be directly synthesized on the biochip.

In accordance with one or more embodiments, the biochips of the present invention are capable of hybridizing to a target sequence under stringent hybridization conditions and attached at spatially defined address on the substrate.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using linkers. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

Exemplary biochips of the present invention include an organized assortment of oligonucleotide probes described above immobilized onto an appropriate platform. In accordance with another embodiment, the biochip of the present invention can also include one or more positive or negative controls. For example, oligonucleotides with randomized sequences can be used as positive controls, indicating orientation of the biochip based on where they are placed on the biochip, and providing controls for the detection time of the biochip when it is used for detecting methylated gene targets from a sample.

Embodiments of the biochip can be made in the following manner. The oligonucleotide probes to be included in the biochip are selected and obtained. The probes can be selected, for example, based on a particular subset target DNA genes of interest. The probes can be synthesized using methods and materials known to those skilled in the art, or they can be synthesized by and obtained from a commercial source, such as GeneScript USA (Piscataway, N.J.).

Each discrete probe is then attached to an appropriate platform in a discrete location, to provide an organized array of probes. Appropriate platforms include membranes and glass slides. Appropriate membranes include, for example, nylon membranes and nitrocellulose membranes. The probes are attached to the platform using methods and materials known to those skilled in the art. Briefly, the probes can be attached to the platform by synthesizing the probes directly on the platform, or probe-spotting using a contact or non-contact printing system. Probe-spotting can be accomplished using any of several commercially available systems, such as the GeneMachines™ OmniGrid (San Carlos, Calif.).

The biochips are scanned, for example, using an Epson Expression 1680 Scanner (Seiko Epson Corporation, Long Beach, Calif.) at a resolution of about 1500 dpi and 16-bit grayscale. The biochip images can be analyzed using Array-Pro Analyzer (Media Cybernetics, Inc., Silver Spring, Md.) software. Because the identity of the target DNA gene probes on the biochip are known, the sample can be identified as including particular target DNA genes when spots of hybridized target DNA genes-and-probes are visualized. Additionally, the density of the spots can be obtained and used to quantitate the identified target DNA genes in the sample.

The methylation state of a disease-associated target DNA gene provides information in a number of ways. For example, a differential methylation state of a cancer-associated gene target compared to a control may be used as a diagnostic that a patient suffers from breast cancer. Methylation states of a cancer-associated gene targets may also be used to monitor the treatment and disease state of a patient. Furthermore, Methylation states of a cancer-associated gene targets may allow the screening of drug candidates for altering a particular expression profile or suppressing an expression profile associated with cancer.

It will be understood by those of ordinary skill in the cancer treatment arts, that the methylation status of the target genes of the present invention can be used to alter the standard treatments given to subjects diagnosed with certain types of cancer.

In accordance with one or more embodiments of the present invention, it will be understood that the types of cancer diagnosis which may be made, using the methods provided herein, is not necessarily limited. For purposes herein, the cancer can be any cancer. As used herein, the term “cancer” is meant any malignant growth or tumor caused by abnormal and uncontrolled cell division that may spread to other parts of the body through the lymphatic system or the blood stream.

It will be understood that the methods of the present invention which determine the methylation state of a sample of DNA are useful in preclinical research activities as well as in clinical research in various diseases or disorders, including, for example, cervical cancer.

The phrase “controls or control materials” refers to any standard or reference tissue or material that has not been identified as having cancer. The methylation state is calculated in part, by comparing the DNA methylation level obtained for the unknown specimen with the level obtained for the standard.

The nucleic acids used as primers in embodiments of the present invention can be constructed based on chemical synthesis and/or enzymatic ligation reactions using procedures known in the art. See, for example, Sambrook et al. (eds.), Molecular Cloning, A Laboratory Manual, 3rd Edition, Cold Spring Harbor Laboratory Press, New York (2001) and Ausubel et al., Current Protocols in Molecular Biology, Greene Publishing Associates and John Wiley & Sons, NY (1994). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleotide sequences used herein are those which hybridize under stringent conditions preferably hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50° C.-70° C.

In accordance with an embodiment, the present invention provides an array of oligonucleotide probes for identifying methylated promoters of target DNA genes in a sample, comprising one or more oligonucleotide probes that each selectively bind methylated loci in a target DNA gene and a platform; wherein the probes are immobilized on the platform; and wherein at least one or more probes selectively bind methylated promoter target DNA genes selected from the group consisting of GGTLA4, CGB5, FKBP6, TRIM74, ZNF516, MICAL-L2, ZAP701, RGS12, SAP130 and INTS1.

In accordance with some embodiments, the array of oligonucleotide probes for identifying methylated promoters of target DNA genes in a sample, at least one or more probes selectively bind methylated promoter target DNA genes selected from the group consisting of FKBP6, INTS1, and ZNF516.

In accordance with some embodiments, the arrays of the present invention further comprise at least one randomly-generated oligonucleotide probe sequence used as a negative control; at least one oligonucleotide sequence derived from a housekeeping gene, used as a negative control for total DNA degradation; at least one randomly-generated sequence used as a positive control; and a series of dilutions of at least one positive control sequence used as saturation controls; wherein at least one positive control sequence is positioned on the array to indicate orientation of the array.

In accordance with an embodiment, the present invention provides a biochip comprising a solid substrate further comprising at least two oligonucleotide probes of any of the arrays described above, which are capable of hybridizing to a target sequence under stringent hybridization conditions and attached at spatially defined address on the substrate.

In accordance with another embodiment, the present invention provides a method for determining the methylation status of one or more target genes in a cervical tissue sample from a subject comprising: a) obtaining a biological sample of comprising DNA from the cervical tissue of the subject; (b) extracting DNA from the sample of a); (c) contacting the DNA from (b) with the any of the arrays described above or the biochip described above; (d) performing an analysis using the array or biochip of c) to determine the methylation of at least one or more target DNA genes obtained from the sample; and (e) comparing the methylation of at least one or more target DNA genes obtained from the sample tissue with the methylation of at least one target DNA gene obtained from a control sample, wherein a detectable increase in the promoter methylation of at least one or more target DNA genes obtained from the sample compared to control wherein when the amount of promoter methylation on at least one or more DNA target genes is greater than the amount of promoter methylation in the control sample, the promoter of the target DNA gene is considered to be methylated.

As used herein, the term “host cell” refers to any type of cell that can contain the viral DNA disclosed herein. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, and the like. In a preferred embodiment, normal cervical epithelium cell line (ECT1 E6/E7), and three cervical cancer cell lines (C-4I, SiHa and C-33A) can be used. In an embodiment, the host cell is preferably a mammalian cell. Most preferably, the host cell is a human cell or human cell line. The host cell can be of any cell type, can originate from any type of tissue, and can be of any developmental stage.

The term “isolated and purified” as used herein means a protein that is essentially free of association with other proteins or polypeptides, e.g., as a naturally occurring protein that has been separated from cellular and other contaminants by the use of antibodies or other methods or as a purification product of a recombinant host cell culture.

The term “biologically active” as used herein means an enzyme or protein having structural, regulatory, or biochemical functions of a naturally occurring molecule.

The term “reacting” in the context of the embodiments of the present invention means placing compounds or reactants in proximity to each other, such as in solution, in order for a chemical reaction to occur between the reactants.

As used herein, the term “treat,” as well as words stemming therefrom, includes diagnostic and preventative as well as disorder remitative treatment.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of diagnosis, staging, screening, or other patient management, including treatment or prevention of cancer in a subject. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

A method of diagnosis is also provided. The method comprises detecting a differential expression level of one, or two or more disease-associated methylation states of a target gene of interest in a biological sample. The sample may be derived from a subject. Diagnosis of a disease state in a subject may allow for prognosis and selection of therapeutic strategy. Further, the developmental stage of cells may be classified by determining temporarily expressed disease-associated methylation states.

### EXAMPLES

Clinical samples. Tissue samples were collected from 2004 to 2008, at the high risk cervical cancer clinic of Doctor Hernan Henriquez Aravena (HHHA) tertiary care regional hospital, in Temuco, Chile. The diagnosis was confirmed by histological examination (biopsy) performed by a team of three pathologists from HHHA. A random set of pathology slides from the study samples was sent for diagnostic confirmatory review to a pathologist at Johns Hopkins University School of Medicine. The protocol for this study was approved by the Institutional Review Boards of the HHHA and the Johns Hopkins University School of Medicine. All normal and CIN samples used in this study were collected by cytobrush. Tumor samples were either cytobrush (18%) or formalin-fixed paraffin-embedded samples that were collected during surgery (82%).

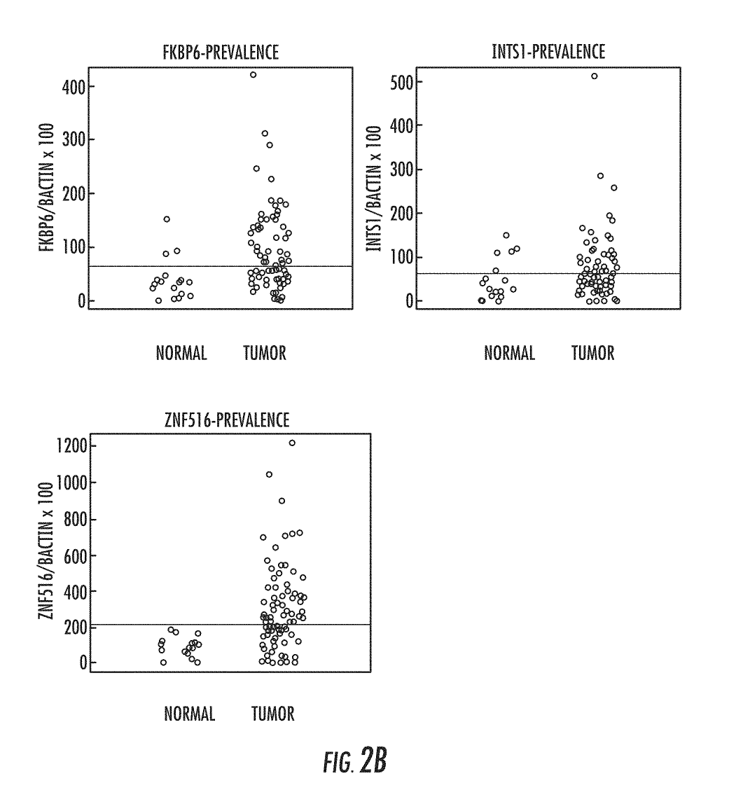

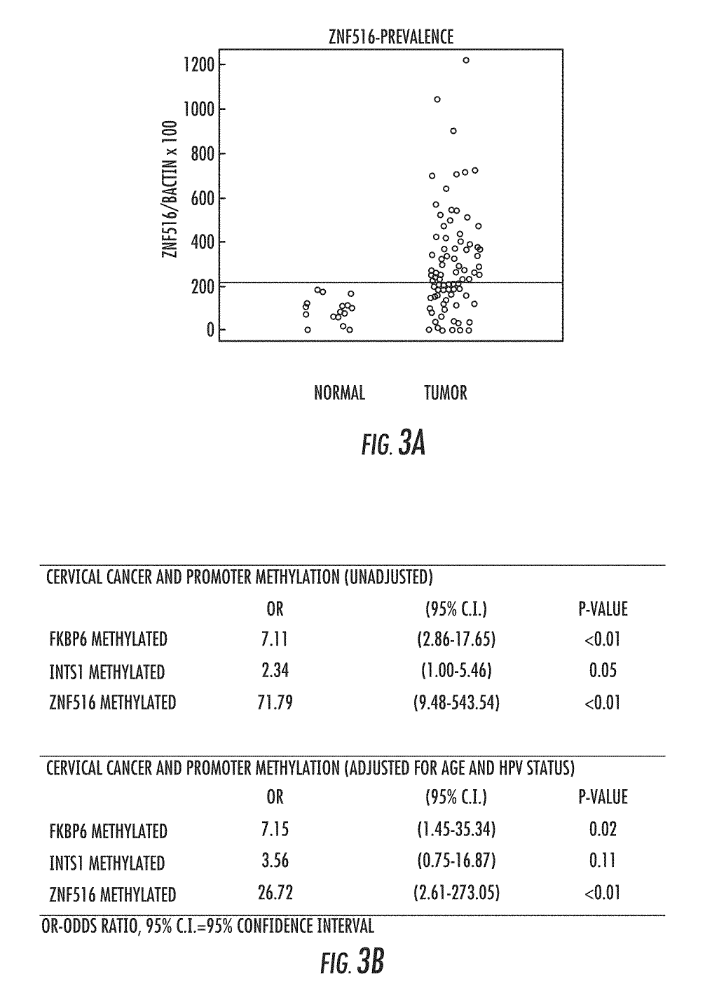

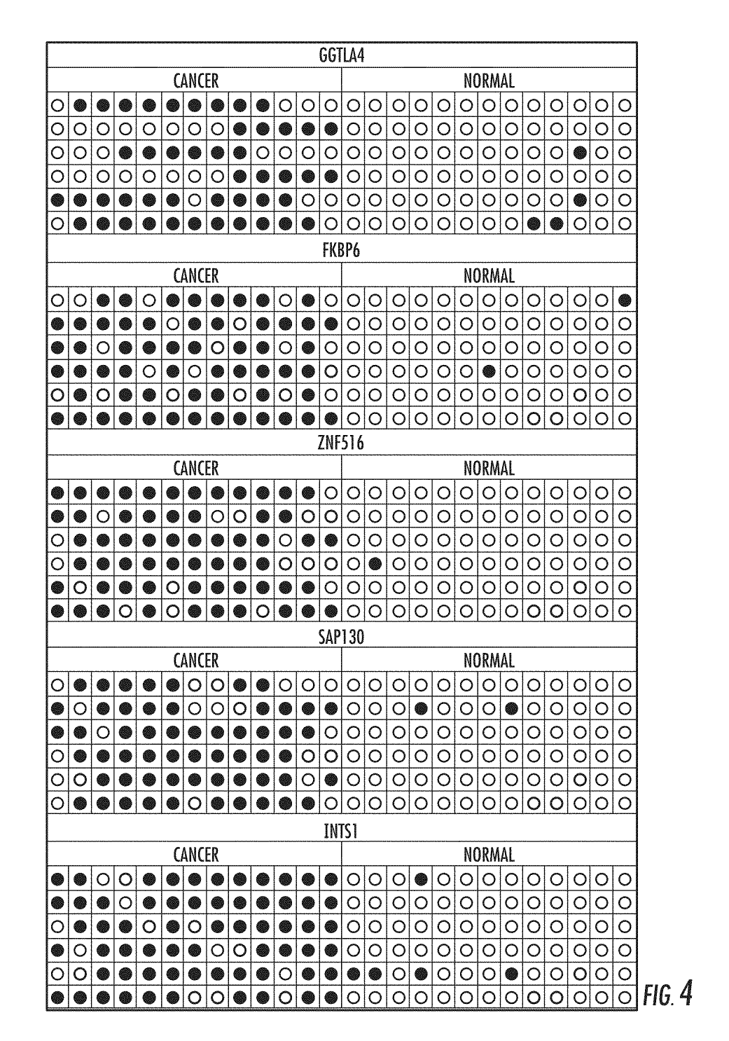

Methylation profiling with MeDIP-chip. A total of 491 genes were shown to be differentially methylated between normal and cervical cancer samples. Based on the selection criteria, the first 10 genes were selected (GGTLA4, CGB5, FKBP6, TRIM74, ZNF516, MICAL-L2, ZAP701, RGS12, SAP130 and INTS1). These genes were amplified in the same samples used to hybridize microarrays and bisulfate sequencing was performed to examine their methylation status. Amplicons sequence was aligned to the gene of interest (http://blast.ncbi.nlm.nih.gov/Blast.cgi) to ascertain their identity. Only five genes were selected as potential biomarkers after to Bisulfite sequence analysis, GGTLA4 (20p11.1), FKBP6 (7q11.23), ZNF516 (18q23), SAP130 (2q14.3) and INTS1 (7p22.3), because these genes had a high percentage of identity (>75%), and were only methylated in cancer samples (FIG. 4).

Internal validation of microaray results. MSP was used to examine the methylation profiles of five genes GGTLA4, FKBP6, ZNF516, INTS1 and SAP130 in the normal and cervical samples hybridized to the microarrays. The 100% of normal samples showed no methylation, where as the cancer samples were methylated in all cases (100%) (FIG. 5).

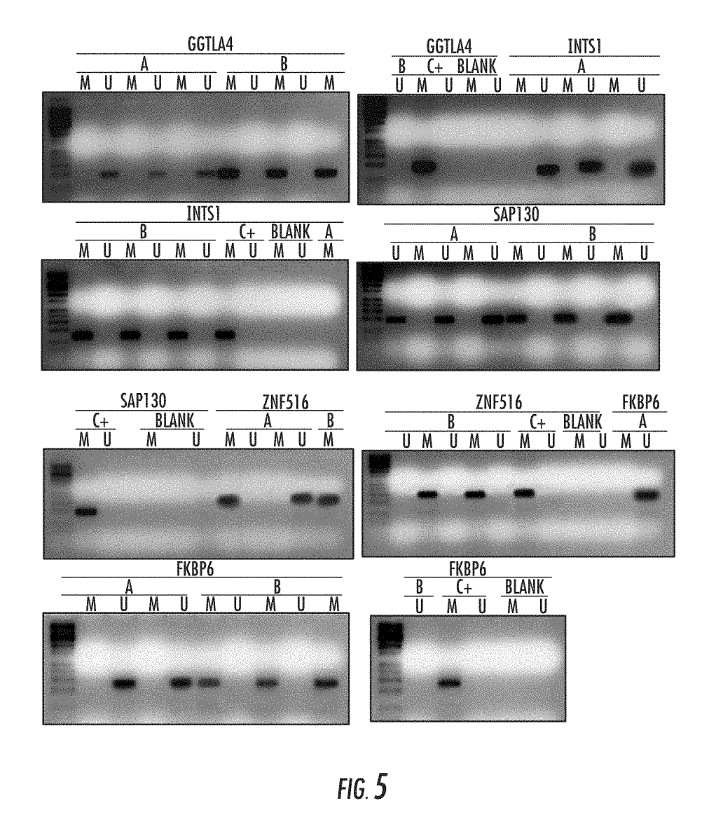

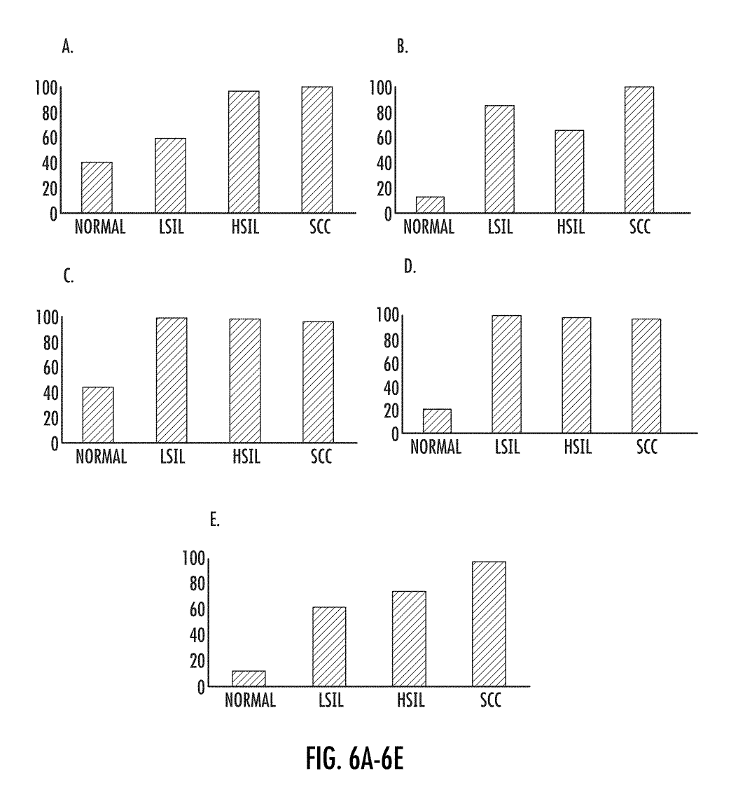

External validation of microarray results. MSP was used to examine the methylation status of GGTLA4, FKBP6, ZNF516, INTS1 and SAP130 in 221 HPV genotyped samples: 25 normal, 66 LSIL, 91 HSIL and 39 CC (FIG. 6).

To determine the methylation status of promoter regions across the genome, twelve normal and seven cervical cancer tissue samples were enriched for methylated DNA with MeDIP and hybridized to oligonucleotide tiled-sequencing arrays (385K CpG Islands plus Promoter arrays, Nimblegen, Wis.). In total, genomic DNA from 37 normal and 120 cancer patients was used for Quantitative Methylation Specific PCR (qMSP) validation of genes that were discovered by MeDIP. Of these patients, 19 normal and 30 cancer patients were randomly selected for inclusion in the Discovery cohort, by using the random selection option in SPSS statistics software (version 19). The remaining 18 normal and 90 cancer samples were selected for the Prevalence cohort. Furthermore, to examine the feasibility of creating a diagnostic panel we examined the promoter methylation status of the best performing candidate tumor suppressor genes in cervical brush biopsies from 137 CIN lesions.

HPV genotyping. HPV detection and genotyping were performed as previously described (J. Clin. Microbiol., 2002; 40:779-87). Reverse Line Blot (RLB) analysis was performed using 38 modified oligoprobes for the analysis. A panel of 36 HPV viral types was used as positive control. HPV 16, 18, 31 and 33 were commercial plasmid clones (ATCC) and the remaining HPV types were provided by Dr. Peter Snijders (VU University Medical Center, Amsterdam, The Netherlands). Negative controls consisted of commercial genomic DNA (Promega, Madison, Wis.) and deionized water.

DNA extraction. Tissue was digested with 1% SDS and 50 μg/ml proteinase K (Boehringer Mannheim, Indianapolis, Ind.) at 48° C. overnight, followed by phenol/chloroform extraction and ethanol precipitation of DNA. The integrity of extracted DNA was verified by a PCR amplification of a 268-bp fragment of the β-globin gene using PCO4 (5′-CAACTTCATCCACGTTCACC-3′) (SEQ ID NO: 1) and GH20 (5′-GAAGAGCCAAGGACAGGTAC-3′) (SEQ ID NO: 2) primers.

MeDIP Discovery workflow. Design, implementation, and validation of the MeDIP-chip experiment workflow was performed in Johns Hopkins University. DNA samples were sent to Johns Hopkins University School of Medicine for MeDIP enrichment prior to shipment to Iceland for sample labeling, array hybridization, and methylation array scanning in Nimblegen's laboratories.

Methylated DNA enrichment and array hybridization. DNA from normal cervical mucosa (n=12) and cervical cancer tissue (n=7) samples, enriched with the methylated DNA immunoprecipitation assay (MeDIP), were hybridized to the 385K CpG Islands plus Promoter oligonucleotide tiling arrays (Nimblegen, Wis.), which quantitatively interrogates 27,728 CpG sites from over 17,000 protein-coding gene promoters. The MagMeDIP kit (Diagenode) was used to enrich DNA with methylated cytosines according to manufacturer's protocol. Genomic DNA (500 ng) was sheared using a water bath sonicator (Bioruptor UCD-200, Diagenode) at “LOW” power setting in the following cycles: (alternating 5 minutes sonication and 2 minutes on ice) for a total sonication time of 15 minutes. Sonicated DNA was then analyzed on a 1.5% agarose gel to ensure that sonicated fragments had an optimal size of 200-1000 bp. Sonicated DNA was denatured for 10 minutes at 95° C. and immunoprecipitated with monoclonal antibody against 5-methylcytidine. The immunoprecipitated methylated DNA (IP) and the input genomic DNA was amplified and purified with the GenomePlex Complete Whole Genome Amplification (WGA) Kit (Sigma-Aldrich) and the QIAquick PCR Purification Kit (Qiagen). IP DNA (2 μg) was labeled with Cy5 fluorophere and the input genomic DNA was labeled with Cy3 fluorophere. Labeled DNA were combined and hybridized to the 385K Human CpG Island-Plus-Promoter Array (Roche-NimbleGen), which represents 28K UCSC-annotated CpG islands and promoter regions for 17K RefSeq genes from the HG18 build.

Differential methylation bioinformatics. The standard Nimblegen algorithms were used to compute the normalized data and identify peaks of enrichment, coinciding with methylated regions. The methylation peak scores for each probe in the methylation arrays were calculated and ranked using the ACME algorithm (Methods Enzymol. 2006; 411:270-82). Next, the data was transformed into a more usable format, i.e. the peaks near known transcription start sites (TSSs) were identified, according to two different cut-offs for the maximal distance between a peak and a TSS: −1000 to +1000, called the standard cut-off; −500 to +500, called the narrow cut-off.

In a first pass analysis at the probe-set level, the cancer specific hypermethylated genes were identified as those genes that had a methylated probe-set in at least one of the primary cancer samples and in none of the normal samples. To maximize the amount of informative loci, this condition was set at a slightly more stringent level: the cancer specific hypermethylated genes were identified as those genes that had a methylated probe-set in 20% or more of the cancer cases. Practically, this is equivalent to at least two samples with methylated probe-sets for a particular gene, out of a total of seven tumor samples. A third more stringent inclusion criteria were implemented to identify cancer specific hypermethylated genes: genes needed to have methylated probe-sets in 100% of cancer and in none of the normal tissues. The probes were then excluded within the candidate gene probe-sets that mapped to chromosomal regions outside of an 800 base pairs window upstream from the transcription start site (TSS). All the candidate genes selected for biomarker validation had methylated probes, within a CPG island located in the promoter region, upstream from the TSS in all the hybridized tumor samples and none in the normal samples hybridized to the arrays. The candidate gene methylated probes were then ranked by methylation peak scores. The genes with the top ten scoring probes were selected for validation with qMSP. The sequences of the methylated probes were utilized to circumscribe the chromosomal regions used to design bisulfate sequencing and MSP primers. All bioinformatics analyses were performed using R version 2.11.1.

Hierarchical clustering analysis and heatmap creation. The log 2 ratio value of all probes on the Nimblegen arrays was used to generate a heatmap based on unsupervised hierarchical clustering with Spotfire DecisionSite (Somerville, Mass.). This clustering was based on the unweighted average method using correlation as the similarity measure and ordering by average values. The color red was selected to represent hypermethylated genes and the color blue to represent hypomethylated genes (data not shown).

Ingenuity Pathway Analysis. Pathway and ontology analysis were performed to identify how differential methylation alters cellular networks and signaling pathways in cervical cancer. A list of RefSeq identifiers for hypermethylated/down-regulated genes was uploaded to the Ingenuity Pathway Analysis program (Redwood City, Calif.), enabling exploration of gene ontology and molecular interaction. Each uploaded gene identifier was mapped to its corresponding gene object (focus genes) in the Ingenuity Pathways Knowledge Base. Core networks were constructed for both direct and indirect interactions using default parameters, and the focus genes with the highest connectivity to other focus genes were selected as seed elements for network generation. New focus genes with high specific connectivity (overlap between the initialized network and gene's immediate connections) were added to the growing network until the network reached a size of 70 nodes. Non-focus genes (those that were not among our differentially methylated input list) that contained a maximum number of links to the growing network were also incorporated. The ranking score for each network was then computed by a right-tailed Fisher's exact test as the negative log of the probability that the number of focus genes in the network is not due to random chance. Similarly, significances for functional enrichment of specific genes were also determined by the right-tailed Fisher's exact test, using all input genes as a reference set.

Differential Methylation events associated to Copy Number Variants. The methylation module of Nexus Copy Number software (BioDiscovery) to identify the cytoband location across the genome of significant hypermethylated events associated to known cancer Copy Number Variants. Nexus uses as input data Nimblegen .gff files, which have the log transformed (log 2) intensity ratios of the red and green channels for each sample after background correction and normalization have been performed. The Running Kolmogorov-Smirnov test (KS) is used to generate methylation peak scores based on the normalized log 2 intensity ratios. KS slides a fixed size window (750 base pairs) along each chromosome to get the methylation calls. The methylation score for any individual probe is based on the distribution of the values of the probes that are within the fixed-sized window, when the window is centered on the probe's midpoint. The methylation score at any individual probe captures how different the distribution of the intensity values that fall in the window are from the overall distribution of intensity values in the array. The probes with a significant methylation score (P<0.05) are plotted along each chromosome and mapped against Copy Number Variation sites known to be altered in cancer.

Validation of in-silico findings with quantitative Methylation Specific PCR (qMSP). Genomic DNA (1 μg) was bisulfite converted with the Epitect Bisulfite kit (Qiagen), according to the manufacturer's instructions and stored at −80° C. Bisulfite conversion was confirmed by amplification of a 280-BP fragment of the β-actin gene. Bisulfite sequence analysis (BS) was performed to determine the methylation status of the normal and tumor tissues used in the tiled-sequencing arrays. Bisulfite-treated DNA was amplified for the 5′ region that included at least a portion of the CpG Island within 800 bp of the proposed transcriptional start site using BS primer sets. The primers for BS were designed to hybridize to regions in the promoter without CpG dinucleotides. PCR products were gel-purified using the QIAquick Gel Extraction Kit (Qiagen) according to the manufacturer's instructions. Each amplified DNA sample was sequenced by the Applied Biosystems 3700 DNA analyzer using nested, forward, or reverse primers and BD terminator dye (Applied Biosystems).

qMSP was used to validate the candidate genes identified with the MeDIP-chip Discovery work flow on a separate cohort of tissue samples from normal and cervical cancer patients. Briefly, bisulfite converted DNA was used as template for fluorescence based real-time PCR, as previously described (Cancer Res. 2008; 68:2661-70). Fluorogenic PCR reactions were carried out in a reaction volume of 10 μl consisting of 300 nmol/l of each primer; 100 μmol/l probe; 0.37.5 units platinum Taq polymerase (Invitrogen); 100 μmol/l of each dATP, dCTP, dGTP, and dTTP; 100 nmol/l ROX dye reference (Invitrogen); 8.3 mmol/l ammonium sulfate; 33.5 mmol/l Trizma (Sigma, St. Louis, Mo.); 3.35 mmol/L magnesium chloride; 5 mmol/L mercaptoethanol; and 0.05% DMSO. Duplicates of three microliters (1.5 μl) of bisulfite-modified DNA solution were used in each real-time methylation-specific PCR (MSP) amplification reaction. Primers and probes were designed to specifically amplify a region in a CpG island in promoters of the genes of interest and the of a reference gene, β-actin as previously described. Primers and probes were tested on positive (genomic methylated bisulfite converted DNA) and negative controls (genomic unmethylated bisulfite converted DNA) to ensure amplification of the desired product and non-amplification of unmethylated DNA, respectively. Primer and probe sequences are provided in Table 1.

TABLE 1
Primer and probe sequences used in the methods of the
present invention.

| | | | Probe 5′/56- | Pro- | |
|---|---|---|---|---|---|
| Gene | | | FAM-/ZEN/- | duct | Tm |
| Name | Forward 5′-3′ | Reverse 5′-3′ | /3IABkFQ/3′ | (BP) | (° C.) |
| BS- | GAGGTTTGTTTGTAGAGGT | CAAAACAACTCTAAAAA | | 397 | 52 |
| GGTLA4 | TC | AATTTTC | | | |
| | (SEQ ID NO: 3) | (SEQ ID NO: 4) | | | |
| BS- | ATAGGGGGAGTTTAAGTAA | CCACTTAACCCAAATAC | | 346 | 54 |
| CGB5 | GG | CCCC | | | |
| | (SEQ ID NO: 5) | (SEQ ID NO: 6) | | | |
| BS- | GTTTTAAAAGTGTTTTTTTT | GAACTCTAAAACTACAA | | 439 | 56 |
| FKBP6 | GTGTTT | AAACCAC | | | |
| | (SEQ ID NO: 7) | (SEQ ID NO: 8) | | | |
| BS- | TTGAGTATGATGGGGTATG | CCCTACTAATAACAAAT | | 443 | 54 |
| TRIM74 | TG | AACTC | | | |
| | (SEQ ID NO: 9) | (SEQ ID NO: 10) | | | |
| BS- | GAGTGTTGTTGGTAGATTG | CTATAAACAATACCAAA | | 347 | 56 |
| ZNF516 | TTG | CCTCAC | | | |
| | (SEQ ID NO: 11) | (SEQ ID NO: 12) | | | |
| BS- | TTTTTTGGAATTTAAGGGTT | GTTGGTTGGGTTGAGTA | | 331 | 54 |
| MICAL- | TTAC | TTATT | | | |
| L2 | (SEQ ID NO: 13) | (SEQ ID NO: 14) | | | |
| BS- | GTTTTGTTTTTTATATTTTTG | CAACCTCCCCCTACCCA | | 414 | 56 |
| ZAP701 | TTTTTG | AAC | | | |
| | (SEQ ID NO: 15) | (SEQ ID NO: 16) | | | |
| BS- | TTTGGGGTTGTTGAAAGAA | CAAACTTTTAAATAACT | | 319 | 56 |
| RGS12 | ATTAT | CCTCCC | | | |
| | (SEQ ID NO: 17) | (SEQ ID NO: 18) | | | |
| BS- | GGGAGGGGTGGGTTGATTC | GCTAACCCCACTCACCC | | 443 | 56 |
| SAP130 | (SEQ ID NO 19) | CC | | | |
| | | (SEQ ID NO: 20) | | | |
| BS- | TTTTTTTTTGTAGTTTTATTT | CCAAAATCACTAAAAAA | | 432 | 54 |
| INTS1 | ATAGC | AAACAAAC | | | |
| | (SEQ ID NO: 21) | (SEQ ID NO: 22) | | | |
| MSP- | TACGACGGTGAGGTACGTA | CAAAAACACAAAAATA | AACGCCAAAC | 241 | 54.2 |
| ZNF516 | TAC | ATACTCGAA | CTCACCGTCGT | | |
| M | (SEQ ID NO 23) | (SEQ ID NO: 24) | ACG | | |
| | | | (SEQ ID NO: 25) | | |
| MSP- | GTATGATGGTGAGGTATGT | CAAAAACACAAAAATA | | 242 | 50 |
| ZNF516 | ATATGA | ATACTCAAA | | | |
| U | (SEQ ID NO: 26) | (SEQ ID NO: 27) | | | |
| MSP- | TTACGTGTTTTATTATGTTT | GAAAAAACACTCATCGT | CGACCCTAAC | 137 | 58 |
| FKBP6 | CGTGC | TTCGTT | CCTCGCGAACT | | |
| M | (SEQ ID NO: 28) | SEQ ID NO: 29) | CTA | | |
| | | | (SEQ ID NO: 30) | | |
| MSP- | ATGTGTTTTATTATGTTTTG | AAAAAAACACTCATCAT | | 135 | 54 |
| FKBP6 | TGTGT | TTCATT | | | |
| U | (SEQ ID NO: 31) | (SEQ ID NO: 32) | | | |
| MSP- | TTGGATATTAAAGGGTGAT | CCGTAATCCTACAAACC | ACGTCCTCCAA | 183 | 55 |
| GGTLA4 | TTTC | CTACG | CTCAACCACTC | | |
| M | (SEQ ID NO 33) | (SEQ ID NO: 34) | CA | | |
| | | | (SEQ ID NO: 35) | | |
| MSP- | TTGGATATTAAAGGGGTGA | TTCCATAATCCTACAAA | | 185 | 52.5 |
| GGTLA4 | TTTTT | CCCTACAT | | | |
| U | (SEQ ID NO: 36) | (SEQ ID NO: 37) | | | |
| MSP- | CGTTAGTTAATAGACGGGA | CTAAATACTACGCCCAA | TCCCGCGCGCT | 189 | 52.5 |
| SAP130 | GGTTC | TAACCG | CTCCGTCTATA | | |
| M | (SEQ ID No: 38) | (SEQ ID NO: 39) | AA | | |
| | | | (SEQ ID NO: 40) | | |
| MSP- | TGTGTTAGTTAATAGATGG | CCTAAATACTACACCCA | | 192 | 55 |
| SAP130 | GAGGTTT | ATAACCAC | | | |
| U | (SEQ ID NO: 41) | (SEQ ID NO: 42) | | | |
| MSP- | CGAAGGGGTTGTTAGTAGT | AAACAAAAAAAATAAC | TATAACCTCCG | 143 | 55 |
| INTS1 M | AGC | CGACGAT | CCCTCCCTCCC | | |
| | (SEQ ID NO: 43) | (SEQ ID NO: 44) | TA | | |
| | | | (SEQ ID NO: 45) | | |
| MSP- | GTGAAGGGGTTGTTAGTAG | AAAAAACAAAAAAAAT | | 147 | 52 |
| INTS1 U | TAGTGT | AACCAACAAT | | | |
| | (SEQ ID NO: 46) | (SEQ ID NO: 47) | | | |
| β-actin | GTGTTTAGGGTTTTTTGTTT | AACCACTCACCTAAATC | ACCACCACCC | 280 | 58 |
| | TTTTT | ATCTTCTC | AACACACAAT | | |
| | (SEQ ID NO: 48) | (SEQ ID NO: 49) | AACAAACACA | | |
| | | | (SEQ ID NO: 50) | | |

BS: Bisulfite sequencing, MSP: Methylation Specific PCR, M: Methylated, U: Unmethylated, BP: base pairs, Tm: melting temperature.

Amplification reactions were carried out in 384-well plates in a 7900 Sequence Detector (Perkin-Elmer Applied Biosystems, Norwalk, Conn.) and were analyzed by SDS 2.3.1 (Sequence Detector System; Applied Biosystems, Norwalk, Conn.). Thermal cycling was initiated with a first denaturation step at 95° C. for 5 minutes, followed by 50 cycles of 95° C. for 15 seconds and 60° C. for one minute. Each plate included patient DNA samples, positive controls (100% Methylated Bisulfite converted DNA, ZymoResearch) and multiple water blanks as non-template controls. Serial dilutions (30-0.003 ng) of this DNA were used to construct a standard curve for each plate. The relative level of methylated DNA for each gene in each sample was determined as a ratio of the amplified gene quantity to the quantity of β-actin multiplied by 100.

In-vitro verification of concurrent hypermethylation and expression downregulation using a pharmacologic unmasking approach. The most significant loci verified by qMSP were then cross-referenced against a report from our group (BMC Med. Genomics, 2008; 1:57) in which we used a relaxation ranking algorithm to identify re-expressed genes in cervical cancer cell lines after treatment with de-methylating agents. Subsequently, we verified the methylation status and expression profile of the most significant loci in a normal cervical epithelium cell line (ECT1 E6/E7), and three cervical cancer cell lines (C-4I, SiHa and C-33A), using real time PCR. All cell lines were obtained from ATCC and used within the first six months after being received in the laboratory.

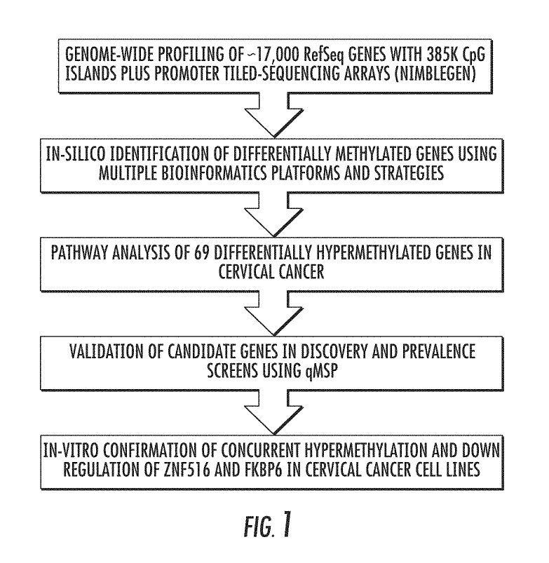

Statistical analysis. All analyses were performed using Stata 11 and SPSS statistics version. The age differences in the Discovery, Prevalence, and Pre-malignant cohorts were compared using the Mann-Whitney U test; differences between socio-economic status, ethnicity and HPV status were analyzed using the chi² test or the Fisher's exact test. The samples were categorized as unmethylated or methylated based on detection of methylation above a threshold set for each gene. Thresholds were determined by ROC curves. To determine predictive accuracy of the methylated genes Spearman Correlation Coefficients, scatter plots, specificity, sensitivity, and Area Under the Curve (N. Engl. J. Med., 2007; 357:1589-97) were used. The Mann-Whitney U test was used to compare methylation levels of different groups. Finally, logistic regression analysis was used to determine the relation between methylation and clinical characteristics. Presence of methylation was used as dependent factor and the various clinical factors were used as independent factors. The association between methylation and clinical diagnosis was also assessed by logistic regression, where clinical diagnosis was used as a response variable, and methylation as a predictive variable. To adjust for age and HPV status, multivariate logistic regression analysis was performed, with clinical diagnosis as dependent and methylation, age, and HPV status as independent factors. Results with a P-value of <0.05 were considered statistically significant. The previously described MeDIP-chip Discovery workflow can be seen in FIG. 1.

Patients' characteristics. The median age of cervical cancer patients was significantly older (51) than normal (41), low (39), and high grade (36) patients (all P<0.01). The ethnic descent of the patients was divided in Mapuche, native Chilean people (24%), and Hispanic/European (76%). Study participants are all public assistance patients receiving income adjusted government health care benefits. The participants were divided into three socioeconomic groups within this subgroup of the Chilean population: indigent; income level ≦US$310; and income level >US$310. PCR and RLB analyses revealed that 80% of the participants (234/294) were HPV positive. As expected, the prevalence of infection with HPV 16 (70%) and HPV 18 (23%) was the highest among cancer patients. In ten of these patients (8%) both HPV 16 and 18 were present.

There were no differences between the Discovery and Prevalence cohort with regard to the normal samples. Cancer patients in the Discovery and Prevalence cohort differed with regard to ethnicity and socio-economic status; cancer patients in the prevalence cohort were more often Mapuche (P=0.02) and more often indigent (P=0.02), than in the Discovery cohort.

### Example 1

Global promoter hypomethylation is a hallmark of cervical cancer. The individual probe methylation values were log-transformed and used to generate a heatmap based on unsupervised hierarchical clustering (data not shown). Unsupervised hierarchical clustering based on the unweighted average method by using correlation as the similarity measure and ordering by log-transformed methylation peak score values. The color red was selected to represent hypermethylated genes and the color blue to represent hypomethylated genes (data not shown. A subset of statistically significant (P<0.01) methylated probes with more than a two-fold change differential methylation value when comparing normal to tumor samples were chosen. Because the empirical P values were calculated genome-wide, adjustment for multiple testing was carried out. The P values were transformed into qvalues, using the Benjamin-Hochberg correction. The probes that were found to have q-values less than 0.05 were deemed to be statistically significant and were included in the final gene list. A visual representation of the significant methylation events in cervical cancer, drawn with the methylation module of Nexus Copy Number software (BioDiscovery) was then prepared (data not shown). The Running Kolmogorov-Smirnov test (KS) was used to generate methylation peak scores based on the normalized log 2 intensity ratios using a fixed size window (750 base pairs) along each chromosome to get the methylation calls. The methylation score for any individual probe is based on the distribution of the values of the probes that are within the fixed-sized window, when the window is centered on the probe's midpoint. The methylation score at any individual probe captures how different the distribution of the intensity values that fall in the window are from the overall distribution of intensity values in the array. The probes with a significant methylation score (P<0.05) are plotted along each chromosome and mapped against Copy Number Variation sites known to be altered in cancer.

The clustering of all CpG loci clearly distinguished between methylation events in normal and cervical cancer tissue. A closer examination of differential methylation in a subset of genes shows a progression to hypermethylation in cervical cancer samples when compared with normal cervical epithelial samples in the genes located at the bottom of the heatmap (data not shown). However, most of the tumor samples showed evidence of global promoter hypomethylation when compared with normal tissue samples, probably related to the sternness characteristics now recognized as a hallmark of tumor cells. This unexpected massive loss of methylation across the promoter regions had not been previously documented in cervical cancer and may potentially be used as a microarray or deep sequencing-based barcode tool to quickly identify tumor from normal samples.

### Example 2

Differential methylation in promoter regions drive oncogenic and phenotypic Pathways. The cellular distribution of the molecular events driven by the 88 hypermethylated and the 86 hypomethylated genes was then examined in cervical cancer. There was a differential distribution for hypermethylation and hypomethylation related cellular events, which may be a reflection of both driving oncogenic transformative events and phenotypic changes resultant from the oncogenic transformation. The functional effects of the gene protein coded by hypermethylated genes seem to be evenly divided between the nucleus, cytoplasm, plasma membrane and extracellular space; whereas, the majority of the molecular events driven by hypomethylated genes seem to be primarily impacting the cytoplasm and the nucleus (data not shown).

### Example 3

Non-stochastic distribution of differential methylation clusters in p and q termini. The cytoband location of the significantly hypermethylated probes across all gene promoters in cervical cancer were identified with Nexus software (data not shown). Notably a large number of differential methylation events seem to be nonstochastically distributed close to the p and q termini of most chromosomes, with the anticipated exception of the X-chromosome, where methylated probes can be seen along the p and q arms. A total of 373 methylated probes had some degree of overlap with known areas of CNV. Most of the methylated probes (78%) showed a 100% CNV overlap in chromosomal regions 381 base pairs long in average (data not shown). However, this is a tiny fraction (0.10%) of the total number of methylated probes (288K). Therefore, CNV overlap with hypermethylated probes does not seem to be an important mechanism in this cervical cancer cohort.

### Example 4

The Nimblegen protocol identified 86 gene probe sets that were hypermethylated in cancer when compared to controls. The distribution of these hypermethylated gene probe sets was examined across chromosomes. The majority of the significantly hypermethylated genes are clustered from chromosome 1 to chromosome 11. Interestingly, the majority of the significantly hypomethylated genes cluster from chromosome 16 to chromosome 22 and on the X chromosome (data not shown).

The functional implication of hypermethylation in cervical cancer was also examined based on known gene function and number of significantly methylated probes per gene that were identified in the in-silico analysis with Nexus. Reassuringly, most of the top ten ranking biological processes play a significant role in oncogenesis: regulation of DNA-dependent transcription; cell differentiation; cell proliferation; chromatin modification; mRNA processing; nucleosome assembly; and insulin receptor signaling pathway.

### Example 5

Ingenuity Pathways Analysis (IPA). Gene networks and canonical pathways representing key genes were identified using the curated Ingenuity Pathways Analysis database as previously described (Int. J. Cancer, 127:2351-9 (2010)). IPA further categorized our data set into functional categories and networks. The Gene ontology analyses of these candidate hypermethylated genes revealed a broad representation of cellular functions in cancer cells: Cell Cycle, Cellular Assembly and Organization, Cellular Function and Maintenance, Cell Death, and Cell Movement, among others (data not shown). Also, the genes are involved in the pathways of NF-kB signaling and DNA methylation and transcriptional repression signaling. This latter observation is of particular interest because the genes we have identified are hypermethylated in the promoter region and/or CpG islands of genes that may be transcriptionally repressed in cervical cancer cells or in precursor lesions.

### Example 6

Validation of candidate genes in Discovery and Prevalence cohorts reveals promoter methylation of ZNF516 and FKBP6 as biomarkers in cervical cancer. More than half of the hypermethylated genes identified by the Nimblegen protocol (60%) were hypermethylated in all cancer samples and not in normal samples. The top-10 genes in this list (GGTLA4, CGB5, FKBP6, TRIM74, ZNF516, MICAL-L2, ZAP701, RGS12, SAP130 and INTS1) were selected for further analysis. Bisulfite sequencing was performed for these genes to examine their methylation status in the same twelve normal and seven cancer patients. Amplicon sequences were aligned to the gene of interest (see, blast.ncbi.nlm.nih.gov/Blast.cgi) to ascertain their identity. Only five genes, GGTLA4, FKBP6, ZNF516, SAP130 and INTS1, were selected as potential biomarkers after bisulfate sequencing, because these genes had a high percentage of identity (>75%), and were only methylated in cancer samples (FIG. 4).

Promoter methylation of FKBP6, INTS1, ZNF516, SAP130, and GGTLA4 was initially determined by qMSP in the Discovery cohort, (19 normal and 30 cancer samples) (FIG. 2A). Correlation with clinical diagnosis, Area Under the Curve, methylation cutoff values, sensitivity, specificity, and the percentage of correctly classified patients are shown in Table 2. Three genes, FKBP6, INTS1, and ZNF516 showed higher methylation in cancer than in normal samples. Using the most optimal cut-off as determined by Receiver Operator Characteristics (ROC) curve, FKBP6 methylation (cutoff 59.58) had a sensitivity of 73% and a specificity of 79%. INTS1 (cut-off 61.34) had a sensitivity and specificity of 50% and 74% respectively. ZNF516 methylation (cut-off 198.68) showed to be the best predictive gene with a sensitivity of 90% and specificity of 95%.

TABLE 2
Predictive Accuracy of FKBP6, INTS1, ZNF516, SAP130 and GGTLA4
with cervical cancer in the discovery (normal n = 19, cancer n = 30) and prevalence cohort
(normal n = 18, cancer n = 90).

| | Spearman | | | Methylation | | | |
|---|---|---|---|---|---|---|---|
| | Correlation | P- | | Cut-off | | | Correctly |
| GENE | Coefficient | value | AUC | Value | Sensitivity | Specificity | Classified |
| Discovery | | | | | | | |
| Cohort | | | | | | | |
| FKBP6 | 0.506 | <0.001 | 0.8 | 59.58 | 73% | 79% | 76% |
| INTS1 | 0.255 | 0.077 | 0.65 | 61.34 | 50% | 74% | 59% |
| ZNF516 | 0.752 | <0.001 | 0.94 | 198.68 | 90% | 95% | 92% |
| SAP130 | −0.552 | <0.001 | 0.29 | 6.94 | 0% | 84% | 33% |
| GGTLA4 | −0.059 | 0.686 | 0.46 | 90.78 | 47% | 47% | 47% |
| Prevalence | | | | | | | |
| cohort | | | | | | | |
| FKBP6 | 0.361 | <0.001 | 0.79 | 59.58 | 58% | 83% | 73% |
| INTS1 | 0.22 | 0.035 | 0.66 | 61.34 | 41% | 76% | 48% |
| ZNF516 | 0.418 | <0.001 | 0.83 | 198.68 | 60% | 100% | 66% |

AUC = area under the curve

Promoter methylation of FKBP6, INTS1 and ZNF516, was then evaluated by qMSP in the Prevalence cohort (18 normal samples and 90 cancer samples) (FIG. 2B). This confirmed the relation between promoter methylation of these genes and cervical cancer, with a sensitivity and specificity of 58% and 83% for FKBP6, 41% and 76% for INTS1, and 60% and 100% for ZNF516 respectively, indicating that ZNF516 methylation has the best predictive value. The ROC analysis of ZNF516 in the Prevalence cohort had an AUC of 0.83.

### Example 7

Promoter methylation is associated with HPV status, age and ethnicity. Univariate logistic regression analysis of various clinical characteristics in all 37 normal and 120 cancer samples revealed that methylation of FKBP6 was related to presence of HPV infection (OR=4.51, 95% C.I.=2.04-9.97, P<0.001) (Table 3). ZNF516 methylation was associated with higher age (OR=1.02, 95% C.I.=1.00-1.05, P=0.03) and HPV infection (OR=11.84, 95% C.I.=4.59-30.57, P<0.001). A borderline significant association was found between methylation of ZNF516 and ethnicity: promoter methylation was less frequently found in Mapuche than in non-Mapuche participants (OR=0.50, 95% C.I.=0.25-1.01, P=0.051).

TABLE 3
Relation between methylation and clinical factors for all normal (n = 37)
and cervical cancer (n = 120) samples

| | | | | Methylation | | |
|---|---|---|---|---|---|---|
| | | | | present | | |
| UM | | M | | | | P- |
| n/total | % | n/total | % | OR | (95% C.I.) | value |

| | | | | | | | |
|---|---|---|---|---|---|---|---|
| FKBP6 | | | | | | | |
| Age (continuous) | | | | | 1.02 | (1.00-1.04) | 0.09 |
| Age (>41) | 41/68 | 60% | 53/70 | 76% | | | |
| Ethnicty (Mapuche) | 24/71 | 34% | 19/75 | 25% | 0.66 | (0.32-1.36) | 0.26 |
| Socio-economic | 39/71 | 55% | 35/75 | 47% | 0.72 | (0.37-1.38) | 0.32 |
| status | | | | | | | |
| (non-indigent) | | | | | | | |
| HPV infection | 40/71 | 56% | 64/75 | 85% | 4.51 | (2.04-9.97) | <0.01 |
| (present) | | | | | | | |
| INTS1 | | | | | | | |
| Age (continuous) | | | | | 1 | (0.98-1.03) | 0.78 |
| Age (>41) | 54/78 | 67% | 39/52 | 75% | | | |
| Ethnicty (Mapuche) | 28/86 | 33% | 15/55 | 27% | 0.78 | (0.37-1.64) | 0.51 |
| Socio-economic | 45/86 | 52% | 28/55 | 51% | 0.94 | (0.48-1.86) | 0.87 |
| status | | | | | | | |
| (non-indigent) | | | | | | | |
| HPV infection | 57/86 | 66% | 43/55 | 78% | 1.82 | (0.84-3.98) | 0.13 |
| (present) | | | | | | | |
| ZNF516 | | | | | | | |
| Age (continuous) | | | | | 1.02 | (1.00-1.05) | 0.03 |
| Age (>41) | 42/74 | 57% | 58/73 | 79% | | | |
| Ethnicty (Mapuche) | 27/74 | 36% | 18/81 | 22% | 0.5 | (0.25-1.01) | 0.05 |
| Socio-economic | 33/74 | 45% | 44/81 | 54% | 1.48 | (0.78-2.78) | 0.23 |
| status | | | | | | | |
| (non-indigent) | | | | | | | |
| HPV infection | 38/74 | 51% | 75/81 | 93% | 11.84 | (4.59-30.57) | <0.01 |
| (present) | | | | | | | |

### Example 8

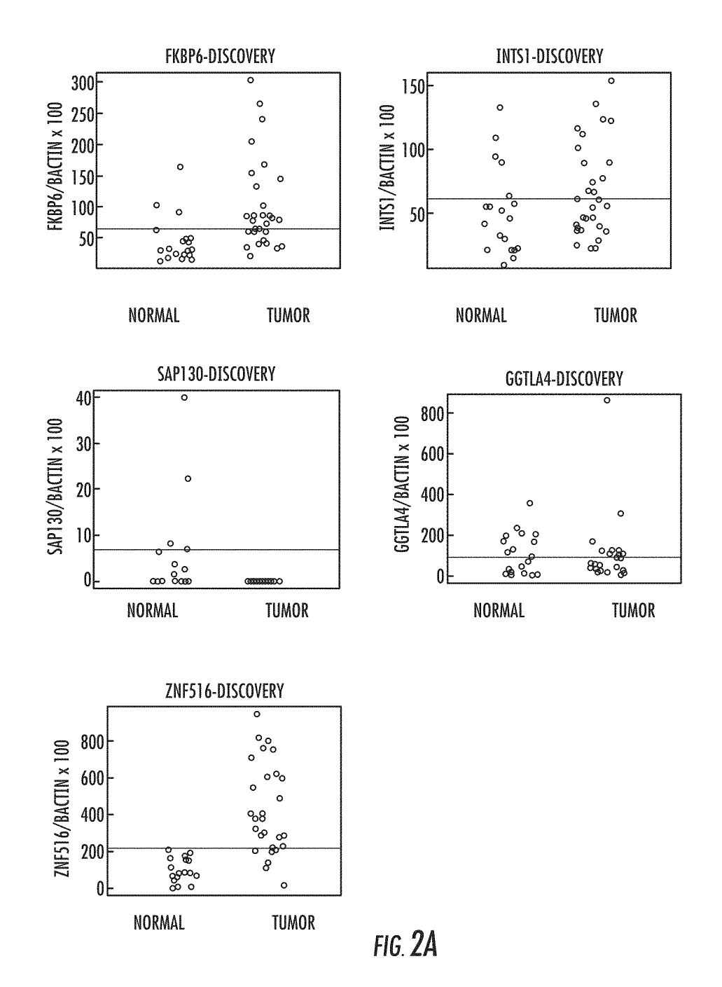

Promoter methylation of ZFN516 is better classifier of normal samples than HPV status. We subsequently examined if promoter methylation of FKBP6 and ZNF516 could correctly classify HPV positive and HPV negative normal and tumor samples. To our surprise promoter methylation of ZNF516 was better than HPV positivity status at classifying normal samples (FIG. 3A). During bivariable analysis we found a significant association between a clinical diagnosis of cancer with both age (OR=1.05, 95% C.I.=1.02-1.08, P<0.01), and with presence of HPV infection (OR=139.78, 95% C.I.=35.81-545.66, P<0.01) (data not shown). We then fitted separate unadjusted and adjusted logistic regression models to examine the association between clinical diagnosis of cancer and promoter methylation of FKBP6, INTS1 and ZNF516 to assess the potential confounding of age and HPV status. This analysis revealed that methylation of FKBP6 (OR=7.15, 95% C.I.=1.45-35.34, P=0.01) and ZNF516 (OR=26.72, 95% C.I.=2.61-273.05, P<0.01) were associated to cervical cancer diagnosis, independently of age and HPV infection (FIG. 3B).

### Example 9

Promoter methylation indicates progression in premalignant cervical lesions. Finally, qMSP for FKBP6, INTS1, and ZNF516 was performed on samples of 137 premalignant lesions. For FKBP6, normal samples (median 32.69) had significantly lower methylation values than CIN lesions (median 95.25, P<0.01). However, the CIN lesions had also higher FKBP6 methylation levels than cervical cancer samples (median 74.54, P<0.01). No difference between INTS1 methylation in cancer (median 55.01) and CIN (median 51.01, P=0.41) was observed, however, in CIN lesions higher methylation values were found than in normal samples (median 40.35, P=0.01). For ZNF516 a gradual increase in methylation levels was observed from normal to cancer (median: normal 84.94, CIN 179.96, cancer 273.75, both P<0.01).

### Example 10

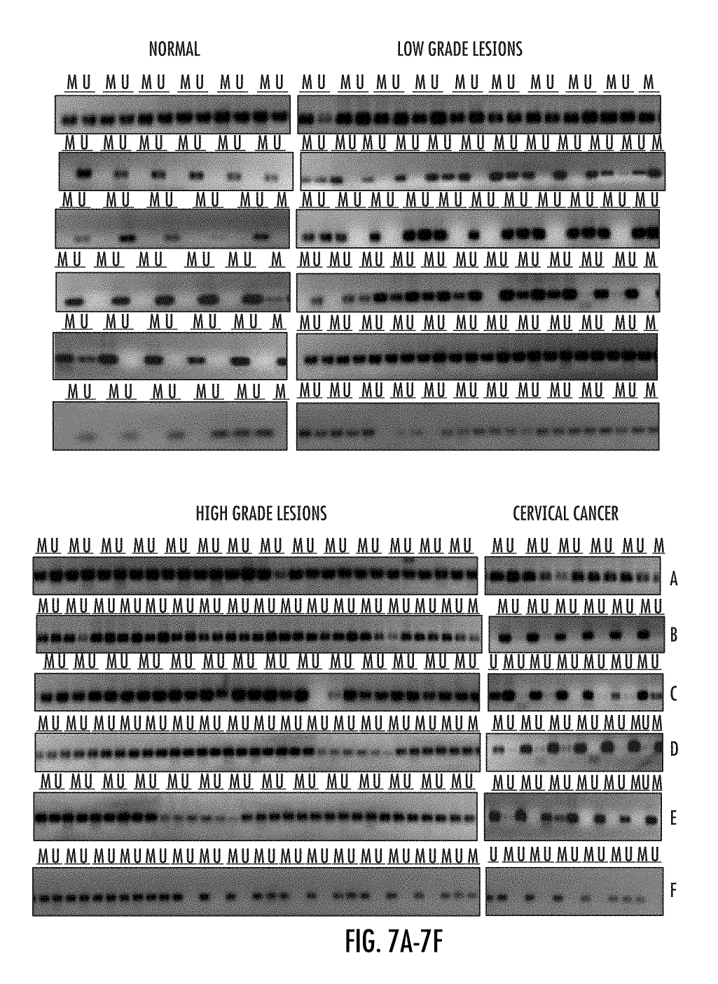

In-vitro verification of concurrent hypermethylation and expression downregulation by pharmacologic unmasking and RT-PCR. Real-time reverse transcriptase-PCR (RT-PCR), and MSP was used to show that ZNF516 and INTS1 are hypermethylated and down-regulated in C-4I and SiHa cervical cancer cell lines (P<0.05) when compared to ECT1 E6/E7 normal cervical epithelium cell lines. C-33A revealed non-significant promoter Hypermethylation and down-regulation of ZNF516 (FIG. 7).

Five candidate genes were identified as differentially methylated with the promoter arrays (FKBP6, INTS1, ZNF516, SAP130, and GGTLA4) and validated by qMSP in the Discovery cohort. This confirmed that FKBP6, INTS1, and ZNF516 were more frequently methylated in cancer than in normal tissues, with ZNF516 methylation being the strongest predictive factor for cervical cancer. FKBP6, and ZNF516 promoter methylation in cervical cancer was subsequently confirmed in the Prevalence cohort, with ZNF516 showing better classification performance than HPV positivity when comparing normal and tumor samples.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for determining the methylation status of one or more target genes in a cervical tissue sample from a subject comprising:
a) obtaining a biological sample of comprising DNA from the cervical tissue of the subject;
(b) extracting DNA from the sample of a);
(c) determining the methylation status of at least one or more target DNA genes selected from the group consisting of ZNF516, INTS1, and FKBP6 obtained from the sample using quantitative Methylation Specific PCR (qMSP) and the primers and probes selected from the group consisting of SEQ ID NOS: 3-50;
(d) comparing the methylation of at least one or more target DNA genes obtained from the sample tissue with the methylation of at least one target DNA gene obtained from a control sample, and
(e) identifying the promoter of the target DNA gene as methylated when the amount of promoter methylation on at least one or more DNA target genes is greater than the amount of promoter methylation in the control sample.

2. A method of diagnosis of cervical cancer in a subject suspected of having cervical cancer comprising:
a) obtaining a biological sample of cervical tissue comprising DNA from the subject;
b) detecting the amount of promoter methylation on at least one or more DNA target sites selected from the group consisting of ZNF516, INTS1, and FKBP6 using quantitative Methylation Specific PCR (qMSP) and the primers and probes selected from the group consisting of SEQ ID NOS: 3-50;
c) comparing the amount of promoter methylation on at least one or more DNA target sites in the sample of the subject to the amount of promoter methylation in a control sample;
d) identifying the subject as having cervical cancer when the amount of promoter methylation on at least one or more DNA target sites is greater than the amount of promoter methylation in the control sample; and
e) identifying an appropriate course of treatment for the subject.

3. The method of claim 2, wherein the subject is suspected of having cervical intraepithelial neoplasia (CIN), and/or low grade squamous intraepithelial lesion (LSIL) and/or high grade squamous intraepithelial lesion (HSIL), or any other abnormal Pap smear or cytological test.


SEQUENCE LISTING (50 sequences)

SEQ ID NO: 1 - DNA, 20 bp, Artificial Sequence
  Description of Artificial Sequence Synthetic primer
caacttcatccacgttcacc

SEQ ID NO: 2 - DNA, 20 bp, Artificial Sequence
  Description of Artificial Sequence Synthetic primer
gaagagccaaggacaggtac

SEQ ID NO: 3 - DNA, 21 bp, Artificial Sequence
  Description of Artificial Sequence Synthetic primer
gaggtttgtttgtagaggttc

SEQ ID NO: 4 - DNA, 24 bp, Artificial Sequence
  Description of Artificial Sequence Synthetic primer
caaaacaactctaaaaaaattttc

SEQ ID NO: 5 - DNA, 21 bp, Artificial Sequence
  Description of Artificial Sequence Synthetic primer
atagggggagtttaagtaagg

SEQ ID NO: 6 - DNA, 21 bp, Artificial Sequence
  Description of Artificial Sequence Synthetic primer
ccacttaacccaaataccccc

SEQ ID NO: 7 - DNA, 26 bp, Artificial Sequence
  Description of Artificial Sequence Synthetic primer
gttttaaaagtgttttttttgtgttt

SEQ ID NO: 8 - DNA, 24 bp, Artificial Sequence
  Description of Artificial Sequence Synthetic primer
gaactctaaaactacaaaaaccac

SEQ ID NO: 9 - DNA, 21 bp, Artificial Sequence
  Description of Artificial Sequence Synthetic primer
ttgagtatgatggggtatgtg

SEQ ID NO: 10 - DNA, 22 bp, Artificial Sequence
  Description of Artificial Sequence Synthetic primer
ccctactaataacaaataactc

SEQ ID NO: 11 - DNA, 22 bp, Artificial Sequence
  Description of Artificial Sequence Synthetic primer
gagtgttgttggtagattgttg

SEQ ID NO: 12 - DNA, 23 bp, Artificial Sequence
  Description of Artificial Sequence Synthetic primer
ctataaacaataccaaacctcac

SEQ ID NO: 13 - DNA, 24 bp, Artificial Sequence
  Description of Artificial Sequence Synthetic primer
ttttttggaatttaagggttttac

SEQ ID NO: 14 - DNA, 22 bp, Artificial Sequence
  Description of Artificial Sequence Synthetic primer
gttggttgggttgagtattatt

SEQ ID NO: 15 - DNA, 27 bp, Artificial Sequence
  Description of Artificial Sequence Synthetic primer
gttttgttttttatatttttgtttttg

SEQ ID NO: 16 - DNA, 20 bp, Artificial Sequence
  Description of Artificial Sequence Synthetic primer
caacctccccctacccaaac

SEQ ID NO: 17 - DNA, 24 bp, Artificial Sequence
  Description of Artificial Sequence Synthetic primer
tttggggttgttgaaagaaattat

SEQ ID NO: 18 - DNA, 23 bp, Artificial Sequence
  Description of Artificial Sequence Synthetic primer
caaacttttaaataactcctccc

SEQ ID NO: 19 - DNA, 19 bp, Artificial Sequence
  Description of Artificial Sequence Synthetic primer
gggaggggtgggttgattc

SEQ ID NO: 20 - DNA, 19 bp, Artificial Sequence
  Description of Artificial Sequence Synthetic primer
gctaaccccactcaccccc

SEQ ID NO: 21 - DNA, 26 bp, Artificial Sequence
  Description of Artificial Sequence Synthetic primer
tttttttttgtagttttatttatagc

SEQ ID NO: 22 - DNA, 25 bp, Artificial Sequence
  Description of Artificial Sequence Synthetic primer
ccaaaatcactaaaaaaaaacaaac

SEQ ID NO: 23 - DNA, 22 bp, Artificial Sequence
  Description of Artificial Sequence Synthetic primer
tacgacggtgaggtacgtatac

SEQ ID NO: 24 - DNA, 25 bp, Artificial Sequence
  Description of Artificial Sequence Synthetic primer
caaaaacacaaaaataatactcgaa

SEQ ID NO: 25 - DNA, 24 bp, Artificial Sequence
  Description of Artificial Sequence Synthetic probe
aacgccaaacctcaccgtcgtacg

SEQ ID NO: 26 - DNA, 25 bp, Artificial Sequence
  Description of Artificial Sequence Synthetic primer
gtatgatggtgaggtatgtatatga

SEQ ID NO: 27 - DNA, 25 bp, Artificial Sequence
  Description of Artificial Sequence Synthetic primer
caaaaacacaaaaataatactcaaa

SEQ ID NO: 28 - DNA, 25 bp, Artificial Sequence
  Description of Artificial Sequence Synthetic primer
ttacgtgttttattatgtttcgtgc

SEQ ID NO: 29 - DNA, 23 bp, Artificial Sequence
  Description of Artificial Sequence Synthetic primer
gaaaaaacactcatcgtttcgtt

SEQ ID NO: 30 - DNA, 24 bp, Artificial Sequence
  Description of Artificial Sequence Synthetic probe
cgaccctaaccctcgcgaactcta

SEQ ID NO: 31 - DNA, 25 bp, Artificial Sequence
  Description of Artificial Sequence Synthetic primer
atgtgttttattatgttttgtgtgt

SEQ ID NO: 32 - DNA, 23 bp, Artificial Sequence
  Description of Artificial Sequence Synthetic primer
aaaaaaacactcatcatttcatt

SEQ ID NO: 33 - DNA, 23 bp, Artificial Sequence
  Description of Artificial Sequence Synthetic primer
ttggatattaaagggtgattttc

SEQ ID NO: 34 - DNA, 22 bp, Artificial Sequence
  Description of Artificial Sequence Synthetic primer
ccgtaatcctacaaaccctacg

SEQ ID NO: 35 - DNA, 24 bp, Artificial Sequence
  Description of Artificial Sequence Synthetic probe
acgtcctccaactcaaccactcca

SEQ ID NO: 36 - DNA, 24 bp, Artificial Sequence
  Description of Artificial Sequence Synthetic primer
ttggatattaaaggggtgattttt

SEQ ID NO: 37 - DNA, 25 bp, Artificial Sequence
  Description of Artificial Sequence Synthetic primer
ttccataatcctacaaaccctacat

SEQ ID NO: 38 - DNA, 24 bp, Artificial Sequence
  Description of Artificial Sequence Synthetic primer
cgttagttaatagacgggaggttc

SEQ ID NO: 39 - DNA, 23 bp, Artificial Sequence
  Description of Artificial Sequence Synthetic primer
ctaaatactacgcccaataaccg

SEQ ID NO: 40 - DNA, 24 bp, Artificial Sequence
  Description of Artificial Sequence Synthetic probe
tcccgcgcgctctccgtctataaa

SEQ ID NO: 41 - DNA, 26 bp, Artificial Sequence
  Description of Artificial Sequence Synthetic primer
tgtgttagttaatagatgggaggttt

SEQ ID NO: 42 - DNA, 25 bp, Artificial Sequence
  Description of Artificial Sequence Synthetic primer
cctaaatactacacccaataaccac

SEQ ID NO: 43 - DNA, 22 bp, Artificial Sequence
  Description of Artificial Sequence Synthetic primer
cgaaggggttgttagtagtagc

SEQ ID NO: 44 - DNA, 23 bp, Artificial Sequence
  Description of Artificial Sequence Synthetic primer
aaacaaaaaaaataaccgacgat

SEQ ID NO: 45 - DNA, 24 bp, Artificial Sequence
  Description of Artificial Sequence Synthetic probe
tataacctccgccctccctcccta

SEQ ID NO: 46 - DNA, 25 bp, Artificial Sequence
  Description of Artificial Sequence Synthetic primer
gtgaaggggttgttagtagtagtgt

SEQ ID NO: 47 - DNA, 26 bp, Artificial Sequence
  Description of Artificial Sequence Synthetic primer
aaaaaacaaaaaaaataaccaacaat

SEQ ID NO: 48 - DNA, 25 bp, Artificial Sequence
  Description of Artificial Sequence Synthetic primer
gtgtttagggttttttgtttttttt

SEQ ID NO: 49 - DNA, 25 bp, Artificial Sequence
  Description of Artificial Sequence Synthetic primer
aaccactcacctaaatcatcttctc

SEQ ID NO: 50 - DNA, 30 bp, Artificial Sequence
  Description of Artificial Sequence Synthetic probe
accaccacccaacacacaataacaaacaca